(19) United States
(12) Patent Application Publication
(10) Pub. No.: US 2017/0311894 A1
(43) Pub. Date: Nov. 2, 2017

(54) INGESTIBLE POWER HARVESTING DEVICE, AND RELATED APPLICATIONS

(71) Applicants: Massachusetts Institute of Technology, Cambridge, MA (US); The Brigham and Women's Hospital, Inc., Boston, MA (US)

(72) Inventors: Phillip Nadeau, Cambridge, MA (US); Dina El-Damak, Cambridge, MA (US); Dean Glettig, Cambridge, MA (US); Yong Lin Kong, Cambridge, MA (US); Niclas Roxhed, Bromma (SE); Robert Langer, Newton, MA (US); Anantha P. Chandrakasan, Belmont, MA (US); Carlo Giovanni Traverso, Newton, MA (US)

(21) Appl. No.: 15/498,268

(22) Filed: Apr. 26, 2017

Related U.S. Application Data
(60) Provisional application No. 62/328,084, filed on Apr. 27, 2016.

(51) Int. Cl. A61B 5/00; A61B 5/01; A61B 5/024; A61B 1/04; H01M 6/04; A61B 5/03; H01M 4/06; A61B 5/145; A61B 5/08; A61B 5/07; H01M 6/34; H01M 4/38

(52) U.S. Cl. CPC A61B 5/6861; H01M 6/34; A61B 5/01; A61B 5/024; A61B 1/041; H01M 6/045; H01M 4/38; H01M 4/06; A61B 5/14539; A61B 5/14503; A61B 5/08; A61B 5/073; A61B 5/036; A61B 2503/40; A61B 2560/0214; H01M 2300/0005

(57) ABSTRACT

Aspects disclosed in the detailed description include an ingestible power harvesting device and related applications. An ingestible power harvesting device includes a cathode electrode and an anode electrode that can catalyze a power generating reaction to generate a direct current (DC) power when surrounded by an acidic electrolyte. The cathode electrode and the anode electrode are coupled to an encapsulated electronic device that includes power harvesting circuitry configured to harvest the DC power and output a DC supply voltage for a prolonged period. In examples discussed herein, the prolonged period is at least five days. The DC supply voltage powers an electronic circuit in the encapsulated electronic device to support a defined in vivo operation (e.g., controlled drug delivery, in vivo vital signs monitoring, etc.). As such, the ingestible power harvesting device can operate in vivo for the prolonged period without requiring an embedded conventional battery.

### PRIORITY APPLICATION

This application claims the benefit of provisional patent application Ser. No. 62/328,084, filed Apr. 27, 2016, the disclosure of which is hereby incorporated herein by reference in its entirety.

### GOVERNMENT SUPPORT

This invention was made with government funds under grant number R01 EB000351 awarded by the National Institutes of Health. The U.S. Government may have certain rights in this invention.

### FIELD OF THE DISCLOSURE

The technology of the disclosure relates generally to ingestible electronic devices.

### BACKGROUND

Ingestible electronics have revolutionized the standard of care for a variety of conditions, and powering these devices is essential for their performance. Current primary cell batteries, though capable of meeting the energy demands of these devices, can be toxic and cause injury to patients.

Thanks to advanced design techniques and technology improvements, average power demand of complementary metal-oxide semiconductor (CMOS) technology has been scaling into the nanowatt (nW) regime. There are also various in vitro studies of short-lived batteries demonstrated with synthetic gastric-like electrolytes. In addition, advances in material design and packaging have demonstrated passive devices that are small enough to be swallowed, but then unfold after ingestion to remain long term, up to seven days in the stomach, for slow-release drug delivery. Moreover, such ingestible devices could one day provide an ingestible non-invasive platform for wireless electronic sensors that perform long-term in vivo vital signs monitoring, without needing to be worn continuously, or implanted under the skin. In this regard, it may be desired to develop alternative battery technologies with focuses on transient electronics that fully disappear at the end of their tasks, electrolytes that are supplied on demand to extend the shelf life of battery cells, material selection for fully biocompatible and biodegradable battery cells, and gastric-magnesium-copper battery cells.

### SUMMARY

Aspects disclosed in the detailed description include an ingestible power harvesting device and related applications. An ingestible power harvesting device, which can be deployed in a gastrointestinal (GI) tract for example, includes a cathode electrode and an anode electrode that can catalyze a power generating reaction to generate a direct current (DC) power when surrounded by an acidic electrolyte (e.g., gastric acid). The cathode electrode and the anode electrode are coupled to an encapsulated electronic device that includes power harvesting circuitry configured to harvest the DC power and output a DC supply voltage for a prolonged period. In examples discussed herein, the prolonged period is at least five days. The DC supply voltage powers an electronic circuit in the encapsulated electronic device to support a defined in vivo operation (e.g., controlled drug delivery, in vivo vital signs monitoring, etc.). As such, the ingestible power harvesting device can operate in vivo for the prolonged period without requiring an embedded conventional battery, thus providing a biocompatible platform for self-powering and bio-safe ingestible medical devices.

In one aspect, an ingestible power harvesting device is provided. The ingestible power harvesting device includes a cathode electrode and an anode electrode configured to catalyze a power generating reaction to generate a DC power between the cathode electrode and the anode electrode in response to being surrounded by an acidic electrolyte. The ingestible power harvesting device also includes an encapsulated electronic device. The encapsulated electronic device includes power harvesting circuitry coupled to the cathode electrode and the anode electrode. The power harvesting circuitry is configured to harvest the DC power generated between the cathode electrode and the anode electrode. The power harvesting circuitry is also configured to output a DC supply voltage based on the harvested DC power for a prolonged period. The encapsulated electronic device also includes an electronic circuit powered by the DC supply voltage and configured to support a defined in vivo operation.

In another aspect, a method for evaluating average power harvested by an ingestible power harvesting device is provided. The method includes deploying an ingestible power harvesting device in a porcine GI tract. The ingestible power harvesting device includes a cathode electrode and an anode electrode configured to catalyze a power generating reaction to generate a DC power between the cathode electrode and the anode electrode in response to being surrounded by an acidic electrolyte. The ingestible power harvesting device also includes an encapsulated electronic device. The encapsulated electronic device includes power harvesting circuitry coupled to the cathode electrode and the anode electrode, the power harvesting circuitry configured to harvest the DC power and output a DC supply voltage based on the harvested DC power. The encapsulated electronic device also includes a radio frequency (RF) transceiver. The method also includes transmitting a plurality of formatted data packets from the RF transceiver within a predetermined duration. The method also includes receiving the plurality of formatted data packets at an ex vivo RF transceiver located within an RF coverage range of the RF transceiver. The method also includes determining an average DC power harvested by the power harvesting circuitry in the predetermined duration based on a count of formatted data packets received at the ex vivo RF transceiver and power consumption associated with transmitting each of the plurality of formatted data packets.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1A is a schematic diagram of an exemplary ingestible power harvesting device that can support defined in vivo operations for a prolonged period of at least five days;

FIG. 1B is a schematic diagram providing an exemplary illustration of an encapsulated electronic device in the ingestible power harvesting device of FIG. 1A;

FIG. 2 is a graph illustrating an exemplary duty cycle of the ingestible power harvesting device of FIG. 1A;

FIG. 3 is a schematic diagram providing an exemplary illustration of a formatted data packet that can be configured to convey information related to the defined in vivo operations supported by the ingestible power harvesting device of FIG. 1A;

FIG. 4 is a schematic diagram of an exemplary system for experimenting with the ingestible power harvesting device of FIG. 1A in a gastrointestinal (GI) tract of a pig;

FIG. 5 is a flowchart of an exemplary process that can be employed for evaluating average power harvested by the ingestible power harvesting device of FIG. 1A in the GI tract of the pig of FIG. 4; and

FIGS. 6A-6C are graphs providing exemplary illustrations of results from experiments conducted in the system of FIG. 4 and according to the process of FIG. 5.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below,” “above,” “upper,” “lower,” “horizontal,” and/or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Aspects disclosed in the detailed description include an ingestible power harvesting device and related applications. An ingestible power harvesting device, which can be deployed in a gastrointestinal (GI) tract for example, includes a cathode electrode and an anode electrode that can catalyze a power generating reaction to generate a direct current (DC) power when surrounded by an acidic electrolyte (e.g., gastric acid). The cathode electrode and the anode electrode are coupled to an encapsulated electronic device that includes power harvesting circuitry configured to harvest the DC power and output a DC supply voltage for a prolonged period. In examples discussed herein, the prolonged period is at least five days. The DC supply voltage powers an electronic circuit in the encapsulated electronic device to support a defined in vivo operation (e.g., controlled drug delivery, in vivo vital signs monitoring, etc.). As such, the ingestible power harvesting device can operate in vivo for the prolonged period without requiring an embedded conventional battery, thus providing a biocompatible platform for self-powering and bio-safe ingestible medical devices.

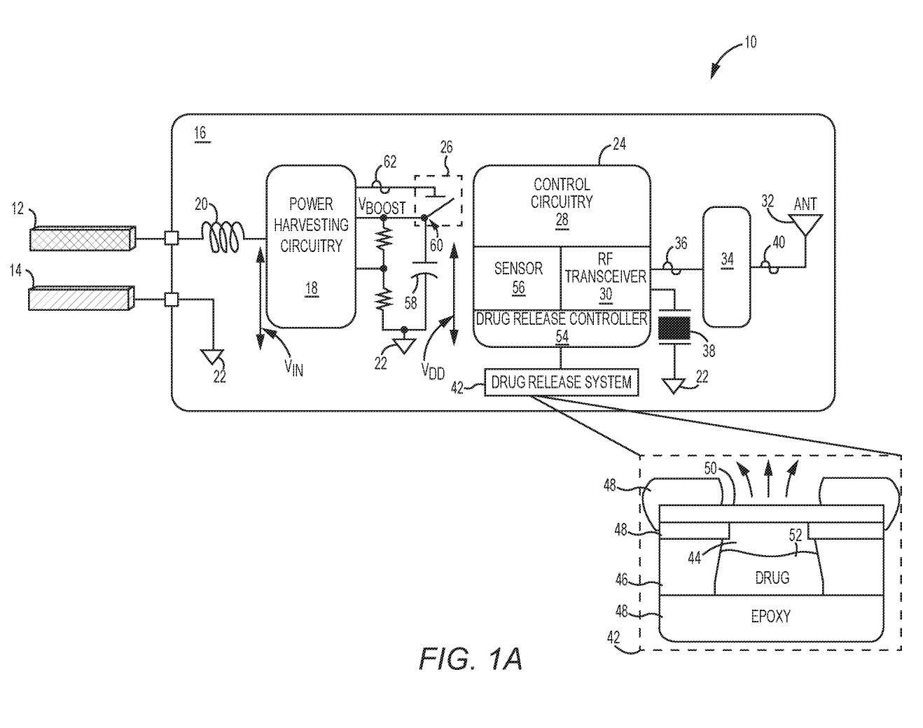

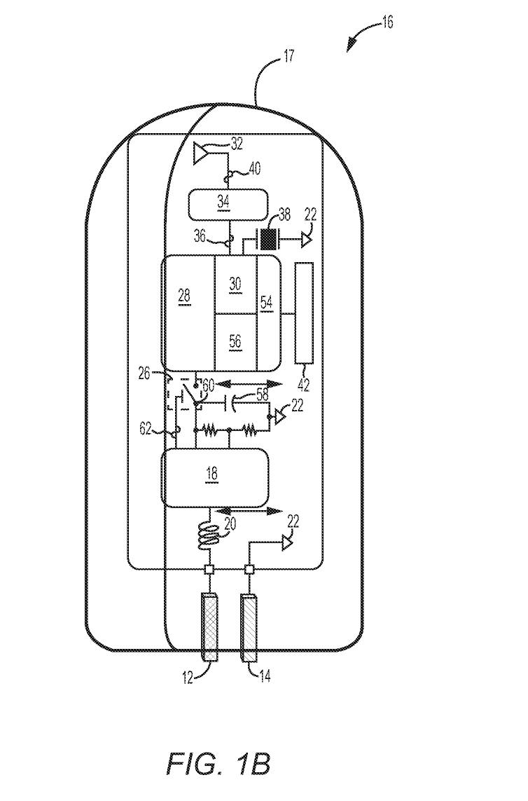

In this regard, FIG. 1A is a schematic diagram of an exemplary ingestible power harvesting device 10 that can support defined in vivo operations for a prolonged period of at least five days. In a non-limiting example, the defined in vivo operations include controlled in vivo drug release, in vivo video capture, in vivo potential-of-hydrogen (pH) measurement, in vivo temperature measurement, in vivo pressure measurement, in vivo heartrate measurement, and in vivo respiration measurement. As discussed in detail below, the ingestible power harvesting device 10 can power the defined in vivo operations for the prolonged period without requiring an embedded conventional battery.

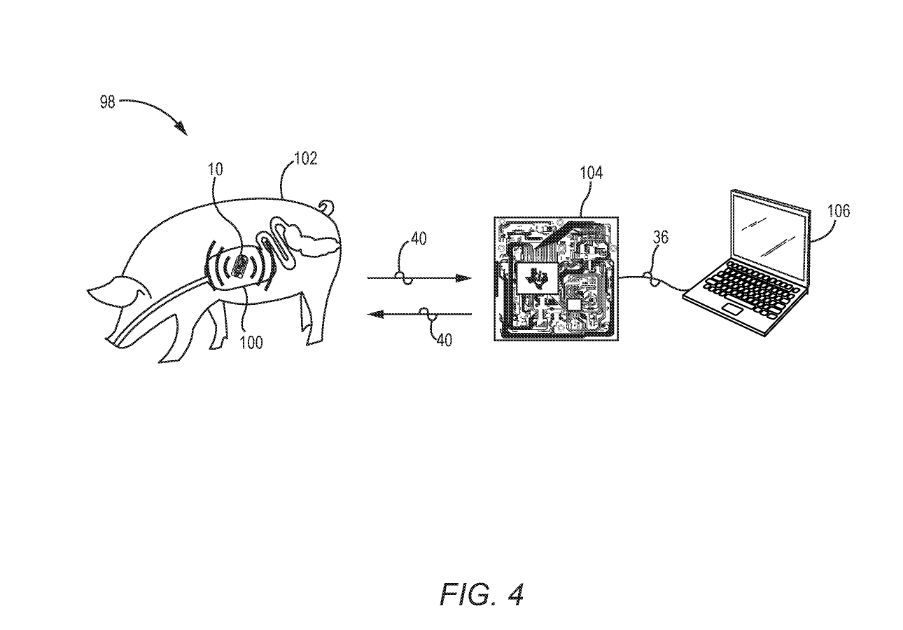

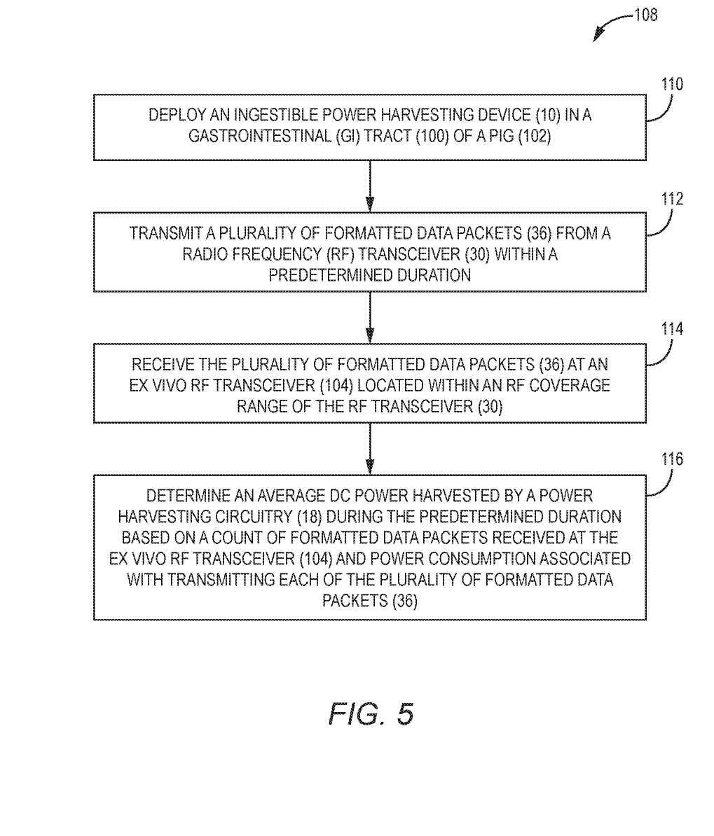

The ingestible power harvesting device 10 includes a cathode electrode 12 and an anode electrode 14. In a non-limiting example, the anode electrode 14 is made of zinc (Zn), and the cathode electrode 12 is made of copper (Cu) metal (e.g., sputtered and patterned copper metal on a substrate). The cathode electrode 12 and the anode electrode 14 can catalyze a power generating reaction to generate DC power between the cathode electrode 12 and the anode electrode 14 when the cathode electrode 12 and the anode electrode 14 are surrounded by an acidic electrolyte. In the exemplary aspects discussed hereinafter, the ingestible power harvesting device 10 is deployed in a GI tract and surrounded by gastric acid in the GI tract. In this regard, the cathode electrode 12 and the anode electrode 14 can catalyze the power generating reaction to generate the DC power in response to being surrounded by the gastric acid in the GI tract. According to an experiment discussed later with reference to FIGS. 4 and 5, when the ingestible power harvesting device 10 is deployed in a porcine GI tract, the anode electrode 14 can generate the DC power of 0.23 microWatts (μW) per square millimeter (mm²) (0.23 μW/mm²) of electrode area for a mean of 6.1 days, longer than the prolonged period.

The ingestible power harvesting device 10 includes an encapsulated electronic device 16, as shown in FIG. 1B. FIG. 1B is a schematic diagram providing an exemplary illustration of the encapsulated electronic device 16 in the ingestible power harvesting device 10 of FIG. 1A. The encapsulated electronic device 16 may be 10 mm in width and 30 mm in length. In a non-limiting example, the encapsulated electronic device 16 can be encapsulated in a capsule 17 by silicone, such as polydimethylsiloxane (PDMS).

With reference back to FIG. 1A, the encapsulated electronic device 16 includes power harvesting circuitry 18, which is coupled to the cathode electrode 12 via an inductor 20. The encapsulated electronic device 16 includes a ground 22. The ground 22 is coupled to the anode electrode 14. The DC power between the cathode electrode 12 and the anode electrode 14 can cause the inductor 20 to induce a DC current. As a result, the DC power between the cathode electrode 12 and the anode electrode 14 can be received by the power harvesting circuitry 18 as a DC input voltage V_{IN}.

The power harvesting circuitry 18, which can be a Texas Instruments BQ25504 ultra low-power boost converter for example, is configured to output a DC supply voltage V_{DD} based on the DC input voltage V_{IN}. In a non-limiting example, the power harvesting circuitry 18 can boost the DC input voltage V_{IN} from 0.2-0.3 volt (V) to the DC supply voltage V_{DD} between 2.2 V and 3.3 V. The power harvesting circuitry 18 is a passive circuitry driven by the DC power between the cathode electrode 12 and the anode electrode 14. In this regard, the power harvesting circuitry 18 can output the DC supply voltage V_{DD} for as long as the cathode electrode 12 and the anode electrode 14 can generate the DC power.

The encapsulated electronic device 16 includes an electronic circuit 24 coupled to the power harvesting circuitry 18 via a switch 26. Accordingly, the electronic circuit 24 can support the defined in vivo operations based on the DC supply voltage V_{DD} generated by the power harvesting circuitry 18. The electronic circuit 24 includes control circuitry 28 and a radio frequency (RF) transceiver 30 that may also be powered by the DC supply voltage V_{DD}. The control circuitry 28 is configured to control the electronic circuit 24 to carry out the defined in vivo operations. The RF transceiver 30 is coupled to an embedded antenna 32 via a matching circuit 34. In one non-limiting example, the RF transceiver 30 can be configured to transmit information related to the defined in vivo operations in one or more formatted data packets 36 via the embedded antenna 32. In this regard, the RF transceiver 30 is powered by the DC supply voltage V_{DD}. The encapsulated electronic device 16 includes a crystal 38 used as a reference for the RF Transceiver 30, which modulates the one or more formatted data packets 36 onto an RF signal 40 for transmission from the embedded antenna 32. In a non-limiting example, the RF signal 40 is transmitted in a 900 MHz RF band. Notably, it may be possible to configure the encapsulated electronic device 16 to store the information related to the defined in vivo operations. As such, the stored information may be post-excreted when the ingestible power harvesting device 10 is discharged. In this regard, the RF transceiver 30 may be externally powered for data read-out.

The RF transceiver 30 may be further configured to receive commands related to the defined in vivo operations and provide the received commands to the control circuitry 28. Accordingly, the control circuitry 28 may be configured to control the electronic circuit 24 to support the defined in vivo operations based on the received commands. In a non-limiting example, the received commands can be used to enable/disable one or more of the defined in vivo operations and/or change a duty cycle of the ingestible power harvesting device 10.

In one non-limiting example, the encapsulated electronic device 16 can be coupled to a drug release system 42, which can be disposed inside or outside the encapsulated electronic device 16. The drug release system 42 may include a drug reservoir 44 enclosed by poly methyl methacrylate (PMMA) 46 and epoxy 48. A gold membrane 50, which may have a thickness of approximately 300 nanometers (300 nm), can be used to seal one or more drugs 52 (e.g., methylene blue) in the drug reservoir 44. The one or more drugs 52 can be released in a controlled fashion from the drug reservoir 44 by corroding the gold membrane 50.

The electronic circuit 24 includes a drug release controller 54 that is powered by the DC supply voltage V_{DD}. The drug release controller 54 is coupled to the drug release system 42 and controls the drug release system 42 to provide a controlled in vivo drug release of the one or more drugs 52 from the drug reservoir 44. The drug release controller 54 may apply at least a portion of the DC supply voltage V_{DD} to create one or more drug release holes in the gold membrane 50, thus allowing the one or more drugs 52 to release from the drug reservoir 44 in the controlled fashion. The RF transceiver 30 may be configured to transmit information related to the controlled in vivo drug release in the one or more formatted data packets 36.

The electronic circuit 24 may include at least one sensor 56 powered by the DC supply voltage V_{DD}. In one non-limiting example, the at least one sensor 56 can be a video sensor configured to support in vivo video capture. In this regard, the RF transceiver 30 may transmit information related to the in vivo video capture (e.g., captured video and/or image) in the one or more formatted data packets 36.

In another non-limiting example, the at least one sensor 56 can be a pH sensor configured to support in vivo pH measurement. In this regard, the RF transceiver 30 may transmit information related to the in vivo pH measurement (e.g., pH value) in the one or more formatted data packets 36.

In another non-limiting example, the at least one sensor 56 can be a temperature sensor configured to support in vivo temperature measurement. In this regard, the RF transceiver 30 may transmit information related to the in vivo temperature measurement (e.g., temperature value) in the one or more formatted data packets 36.

In another non-limiting example, the at least one sensor 56 can be a pressure sensor configured to support in vivo pressure measurement. In this regard, the RF transceiver 30 may transmit information related to the in vivo pressure measurement (e.g., pressure value) in the one or more formatted data packets 36.

In another non-limiting example, the at least one sensor 56 can be a heartrate sensor configured to support in vivo heartrate measurement. In this regard, the RF transceiver 30 may transmit information related to the in vivo heartrate measurement (e.g., heartrate value) in the one or more formatted data packets 36.

In another non-limiting example, the at least one sensor 56 can be a respiration sensor configured to support in vivo respiration measurement. In this regard, the RF transceiver 30 may transmit information related to the in vivo respiration measurement (e.g., respiration value) in the one or more formatted data packets 36.

The power harvesting circuitry 18 is coupled to a capacitor 58, which may have a capacitance of 220 microfarad (220 μF). One end of the capacitor 58 is coupled to the switch 26 at a coupling point 60, and another end of the capacitor 58 is coupled to the ground 22. When the ingestible power harvesting device 10 is first deployed in the GI tract, the power harvesting circuitry 18 is not activated immediately. As such, the capacitor 58 is pulled down to the ground 22 and a voltage V_{C} at the coupling point 60 would be 0 V. As the power harvesting circuitry 18 starts to receive the DC input voltage V_{IN}, the capacitor 58 is gradually charged to ramp up the voltage V_{C} at the coupling point 60 to an internal threshold of the power harvesting circuitry 18. Once the voltage V_{C} at the coupling point 60 reaches the internal threshold, the power harvesting circuitry 18 sets an OK signal 62. Once the OK signal 62 is set, the switch 26 is activated and connects the coupling point 60 to the electronic circuit 24. Accordingly, the capacitor 58 is continuously charged to eventually raise the voltage V_{C} at the coupling point 60 to the DC supply voltage V_{DD}.

In a non-limiting example, the switch 26 is a metal-oxide semiconductor field-effect transistor (MOSFET) switch having a gate electrode coupled to the coupling point 60. The MOSFET switch can be turned on when the voltage V_{C} at the coupling point 60 is greater than or equal to a threshold voltage, and turned off when the voltage V_{C} at the coupling point 60 is lower than the threshold voltage. In this regard, when the capacitor 58 is charged to raise the voltage V_{C} at the coupling point 60 above the threshold voltage, the MOSFET switch is turned on to couple the electronic circuit 24 to the power harvesting circuitry 18. Subsequently, the capacitor 58 begins to discharge the DC supply voltage V_{DD} to power the electronic circuit 24 to perform the defined in vivo operations. As the capacitor 58 discharges, the voltage V_{C} at the coupling point 60 begins to decrease. When the voltage V_{C} falls below a threshold voltage defined by the power harvesting circuitry 18, the OK signal 62 is de-asserted and the MOSFET switch is turned off, thus decoupling the electronic circuit 24 from the power harvesting circuitry 18. The power harvesting circuitry 18 once again charges the capacitor 58 to raise the voltage V_{C} and eventually enables the OK signal 62 again when the voltage V_{C} rises above the threshold voltage. Once the OK signal 62 is set, the MOSFET switch is enabled once again, and couples the capacitor 58 to the electronic circuit 24. The capacitor 58 once again discharges the DC supply voltage V_{DD}, and the MOSFET switch is once again turned off when the voltage V_{C} falls below the threshold voltage. The cycle of charging and discharging the capacitor 58 repeats until the ingestible power harvesting device 10 reaches its lifespan or is discharged from the GI tract.

In this regard, the power harvesting circuitry 18 outputs the DC supply voltage V_{DD} to the electronic circuit 24 periodically. Accordingly, the electronic circuit 24 performs the defined in vivo operations on a periodic basis as well.

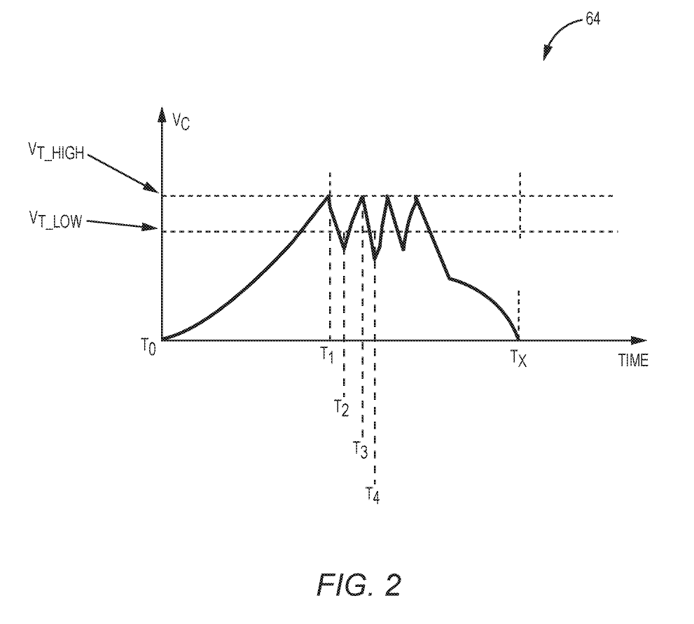

FIG. 2 is a graph illustrating an exemplary duty cycle 64 of the ingestible power harvesting device 10 of FIG. 1A. At time T₀, the ingestible power harvesting device 10 is deployed in the GI tract. As previously discussed, the voltage V_{C} at the coupling point 60 is 0 V. At time T₁, the power harvesting circuitry 18 completes the startup phase and raises the voltage V_{C} to the DC supply voltage V_{DD} that is above the threshold voltage of the MOSFET switch. In a non-limiting example, the DC supply voltage V_{DD} is between a boost circuitry low threshold voltage V_{T}_{_}_{LOW} (e.g., 3.0 V) and a boost circuitry high threshold voltage V_{T}_{_}_{HIGH} (e.g., 3.2 V). The MOSFET switch is thus turned on to couple the electronic circuit 24 to the power harvesting circuitry 18, and the voltage V_{C} at the coupling point 60 starts to decrease as the capacitor 58 is discharged. At time T₂, the voltage V_{C} at the coupling point 60 drops below the threshold voltage defined by the power harvesting circuitry 18. As a result, the MOSFET switch is turned off to decouple the electronic circuit 24 from the power harvesting circuitry 18, and the power harvesting circuitry 18 begins to recharge the capacitor 58. At time T₃, the voltage V_{C} at the coupling point 60 once again turns on the MOSFET switch. At time T₄, the MOSFET switch is once again turned off as the voltage V_{C} falls below the threshold voltage of the MOSFET switch. Finally at time T_{X}, the ingestible power harvesting device 10 reaches the end of the lifecycle (e.g., being discharged from the GI tract).

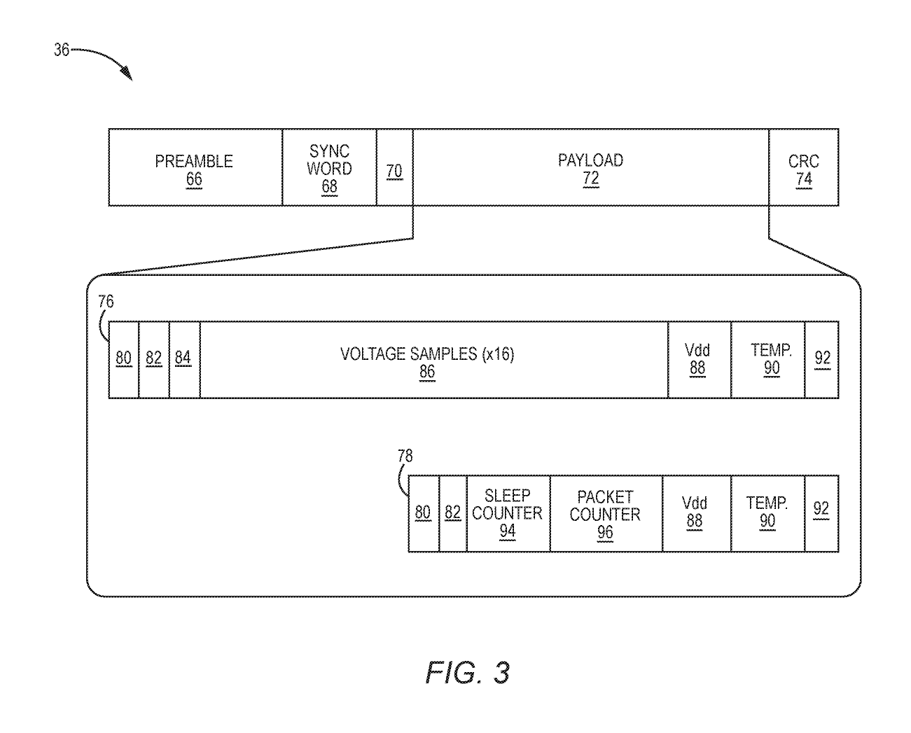

As discussed earlier with reference to FIG. 1A, the RF transceiver 30 may be configured to transmit the information related to the defined in vivo operations in the one or more formatted data packets 36. In this regard, FIG. 3 is a schematic diagram providing an exemplary illustration of a formatted data packet 36 that can be configured to convey the information related to the defined in vivo operations.

The formatted data packet 36 includes a preamble field 66, a sync word field 68, a length field 70, a payload field 72, and a cyclic redundancy check (CRC) field 74. In a non-limiting example, the payload field 72 is formatted to convey an electrical characterization sub-packet 76 and/or a harvesting demonstration sub-packet 78. It shall be appreciated that the payload field 72 can be further formatted to carry other types of sub-packets, such as a configuration/control command sub-packet, an in vivo operation result sub-packet, etc.

The electrical characterization sub-packet 76 includes a board identification (BID) field 80, a packet type identification (PID) field 82, and a resistance identification field 84. The electrical characterization sub-packet 76 also includes a voltage sample field 86, an input voltage field 88, and a temperature value field 90. The voltage sample field 86 may be configured to convey a reading of the DC supply voltage V_{DD}, and the input voltage field 88 may be configured to convey a reading of the DC input voltage V_{IN}. The temperature value field 90 may be configured to convey a value of the in vivo temperature measurement, the in vivo pH measurement, the in vivo heartrate measurement, the in vivo pressure measurement, or the in vivo respiration measurement. The electrical characterization sub-packet 76 also includes a reserved field 92. Notably, the electrical characterization sub-packet 76 can be reformatted to convey any other type of information related to the ingestible power harvesting device 10.

The harvesting demonstration sub-packet 78 includes the BID field 80, the PID field 82, the input voltage field 88, the temperature value field 90, and the reserved field 92. The harvesting demonstration sub-packet 78 also includes a sleep counter field 94 and a packet counter field 96. The sleep counter field 94 may be configured to convey duty cycle information of the ingestible power harvesting device 10. The packet counter field 96 can be configured to convey a value of a transmitted packet counter. As is further discussed with reference to FIG. 5, the transmitted packet counter can be included in the encapsulated electronic device 16 to help detect and mitigate the impact of a lost data packet.

Characterization and performance of the ingestible power harvesting device 10 of FIG. 1A can be determined based on experiments conducted by deploying the ingestible power harvesting device 10 in a porcine GI tract. In this regard, FIG. 4 is a schematic diagram of an exemplary system 98 for experimenting with the ingestible power harvesting device 10 of FIG. 1A in a GI tract 100 of a pig 102.

The RF transceiver 30 is configured to transmit the one or more formatted data packets 36 in the RF signal 40. An ex vivo RF transceiver 104 located within an RF coverage range (e.g., 2-3 meters) of the RF transceiver 30 is configured to receive the RF signal 40 and provide the one or more formatted data packets 36 carried in the RF signal 40 to a personal computer (PC) 106 for analysis and display. As previously discussed, the one or more formatted data packets 36 may contain such information related to the controlled in vivo drug release, the in vivo video capture, the in vivo pH measurement, the in vivo temperature measurement, the in vivo pressure measurement, the in vivo heartrate measurement, and the in vivo respiration measurement.

The PC 106 may configure and/or control the ingestible power harvesting device 10 by including configuration/control commands in the one or more formatted data packets 36. The ex vivo RF transceiver 104 receives and modulates the one or more formatted data packets 36 onto the RF signal 40 for transmitting to the RF transceiver 30. As previously discussed, the configuration/control commands may be used to enable/disable one or more of the defined in vivo operations and/or change the duty cycle of the ingestible power harvesting device 10.

The experiment in the system 98 can be conducted according to a process. In this regard, FIG. 5 is a flowchart of an exemplary process 108 that can be employed for evaluating an average power harvested by the ingestible power harvesting device 10 of FIG. 1A in the GI tract 100 of the pig 102 of FIG. 4.

To start the experiment in the system 98 of FIG. 4, the ingestible power harvesting device 10 is first deployed in the GI tract 100 of the pig 102 (block 110). The RF transceiver 30 in the electronic circuit 24 is configured to transmit a plurality of formatted data packets 36 within a predetermined duration (block 112). The ex vivo RF transceiver 104 in the system 98, which is located within the RF coverage range of the RF transceiver 30, receives the plurality of formatted data packets 36 (block 114). The ex vivo RF transceiver 104 is configured to provide the plurality of received formatted data packets 36 to the PC 106. The PC 106 is configured to determine an average DC power harvested by the power harvesting circuitry 18 during the predetermined duration based on a count of formatted data packets received at the ex vivo RF transceiver 104 and power consumption associated with transmitting each of the plurality of formatted data packets 36 (block 116).

Notably, the RF transceiver 30 is the dominant energy consumer in the ingestible power harvesting device 10. As such, if power consumed by the RF transceiver 30 for transmitting each of the plurality of formatted data packets 36 can be predetermined, it may be possible to estimate the average DC power harvested by the power harvesting circuitry 18 based on the count of the formatted data packets received at the ex vivo RF transceiver 104 in the predetermined duration, as shown in equation Eq. 1 below.

$${P}_{sysavg} = \frac{1}{{T}_{window}} \times {\sum }_{i = 1}^{M} {E}_{pkt} ( {V}_{DD} [ i ] ) \qquad ( Eq . 1 )$$

In the equation Eq. 1 above, P_{sysavg} represents the average DC power harvested by the power harvesting circuitry 18, T_{window} represents the predetermined duration, Eₚₖₜ(V_{DD}) represents the power consumed by the RF transceiver 30 for transmitting each of the plurality of formatted data packets 36 in the predetermined duration T_{window} as a function of the DC supply voltage V_{DD}, and M represents the count of the formatted data packets received at the ex vivo RF transceiver 104.

In a non-limiting example, the control circuitry 28 in the ingestible power harvesting device 10 can be configured to implement a transmitted packet counter (e.g., a software counter) to keep track of the plurality of formatted data packets 36 transmitted by the RF transceiver 30. As previously discussed with reference to FIG. 3, the harvesting demonstration sub-packet 78 in the formatted data packet 36 includes the packet counter field 96. As such, the control circuitry 28 can embedded the present value of the transmitted packet counter in each of the plurality of formatted data packets 36 before the plurality of formatted data packets 36 is transmitted from the RF transceiver 30.

The ex vivo RF transceiver 104 receives the plurality of formatted data packets 36 transmitted from the RF transceiver 30 in the ingestible power harvesting device 10 and provides the plurality of formatted data packets 36 to the PC 106. The PC 106 can thus determine the count of the the formatted data packets received at the ex vivo RF transceiver 104, which is represented by M in the equation Eq. 1 above, based on a maximum packet counter value in the packet counter field 96 conveyed in the plurality of formatted data packets 36 received by the ex vivo RF transceiver.

In another non-limiting example, the power consumed by the RF transceiver 30 for transmitting each of the plurality of formatted data packets 36 (Eₚₖₜ(V_{DD})) can be determined based on a laboratory experiment. For example, a laboratory power supply can be connected to a test RF transmitter having similar gain and peak power as the RF transceiver 30. The test RF transmitter may be configured to transmit an experimental data packet having an identical packet length (e.g. 176 bits) as the formatted data packet 36. In addition, the test RF transmitter may be configured to transmit the experimental data packet at similar data rate (e.g., 50 kbps) and power (e.g., 10 dBm) as the RF transceiver 30. Thus, by measuring the power consumption associated with transmitting the experimental data packet, it may be possible to predetermine the power consumed by the RF transceiver 30 for transmitting each of the plurality of formatted data packets 36 (Eₚₖₜ(V_{DD})).

According to previous discussions with reference to FIGS. 1A and 2, the power harvesting circuitry 18 begins outputting the DC supply voltage V_{DD} to the electronic circuit 24 periodically after completing the startup phase. Accordingly, the control circuitry 28 can control the RF transceiver 30 to transmit the plurality of formatted data packets 36 at a packet rate depending on the DC supply voltage V_{DD}. In this regard, the control circuitry 28 regulates the packet rate by periodically sampling the DC supply voltage V_{DD} output by the power harvesting circuitry 18. If the sampled DC supply voltage V_{DD} is below 3.0 V, for example, the electronic circuit 24 may enter a low-energy sleep mode for 4 seconds (4 s), for example, before attempting to sample the DC supply voltage V_{DD} again. If the sampled DC supply voltage V_{DD} is above 3.0 V, the RF transceiver 30 can transmit one of the plurality of formatted data packet 36 to the ex vivo RF transceiver 104. Understandably, transmission of the formatted data packet 36 would cause an instantaneous drop in the DC power because wireless communication by the RF transceiver 30 is the dominant energy consumer in the ingestible power harvesting device 10. Afterwards, the control circuitry 28 samples the DC supply voltage V_{DD} after 0.5 second, for example, to determine whether to transmit another one of the plurality of formatted data packets 36 or to reenter the low-energy sleep mode for 4 s.

As previously discussed with reference to FIG. 3, the harvesting demonstration sub-packet 78 in the formatted data packet 36 also includes the temperature value field 90. In this regard, the plurality of formatted data packets 36 transmitted from the RF transceiver 30 may also include temperature measurement in the GI tract 100 of the pig 102.

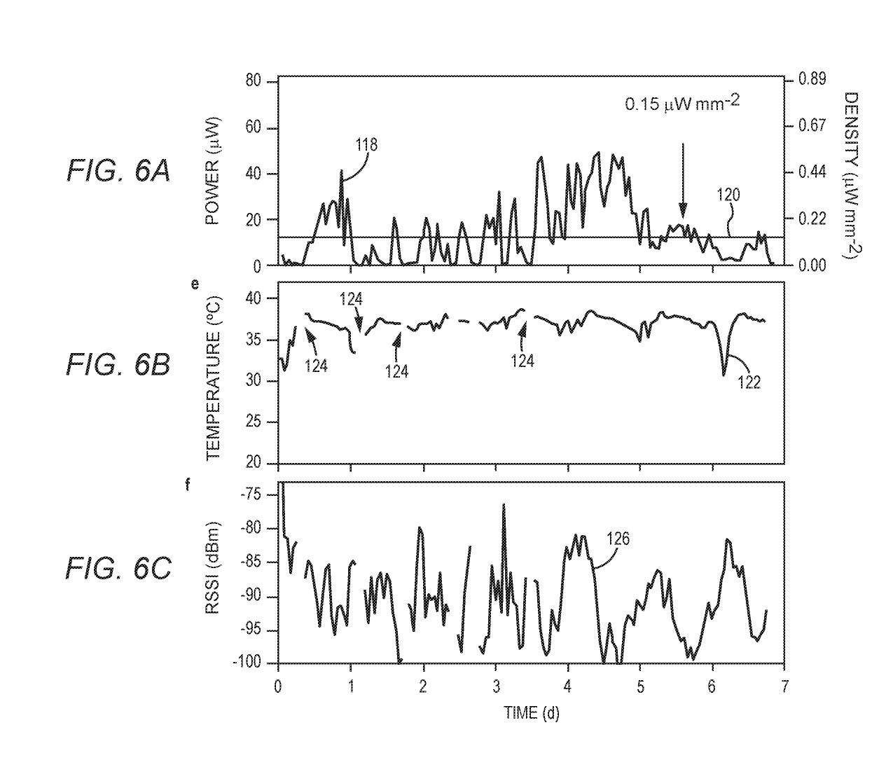

Results of the experiment conducted in the system 98 of FIG. 4 based on the process 108 of FIG. 5 can be graphically illustrated. In this regard, FIGS. 6A-6C are graphs providing exemplary illustrations of the results from the experiment conducted in the system 98 of FIG. 4 and according to the process 108 of FIG. 5.

FIG. 6A includes an estimated DC power curve 118 and an average DC power curve 120. The estimated DC power curve 118 illustrates estimated DC power corresponding to each of the plurality of formatted data packets 36 received by the ex vivo RF transceiver 104 during the predetermined duration T_{window}. The average DC power curve 120 illustrates the average DC power harvested by the power harvesting circuitry 18 in accordance with the equation Eq. 1. In a non-limiting example, the predetermined duration T_{window} is 0.5 hour. Accordingly, the average DC power harvested by the power harvesting circuitry 18 is approximately 0.15 μW/mm².

FIG. 6B includes a temperature measurement curve 122 illustrating the temperature measurement in each of the plurality of formatted data packets 36 received by the ex vivo RF transceiver 104. In a non-limiting example, temperature measurements are received every 12 seconds. Notably, the temperature measurement curve 122 also includes a plurality of gaps 124. In a non-limiting example, the plurality of gaps 124 indicates that the electronic circuit 24 enters the low-energy sleep mode (4 s) as a result of the DC supply voltage V_{DD} being lower than the voltage (e.g., 3 V) required to transmit the plurality of formatted data packets 36.

FIG. 6C includes a received signals strength indicator (RSSI) curve 126. The RSSI curve 126 illustrates respective RSSIs of the plurality of formatted data packets 36 received at the ex vivo RF transceiver 104.

In a non-limiting example, the experiment conducted in the system 98 of FIG. 4 and according to the process 108 of FIG. 5 is repeated in three pigs using three different ingestible power harvesting devices to help provide more accurate results. The results of the three experiments conducted in the three different pigs are summarized in the Table 1 below.

TABLE 1

| | Experiments | | | |
|---|---|---|---|---|
| | 1 | 2 | 3 | Average |
| Operating time (days) | 6.82 | 6.61 | 4.73 | 6.05 |
| Average packet interval (second) | 15.7 | 14.0 | 6.8 | 12.17 |
| Average power density (μW/mm²) | 0.15 | 0.18 | 0.36 | 0.23 |
| Energy delivered (μW * h/mm²) | 24.5 | 28.2 | 40.5 | 31.07 |
| Average RSSI (dBm) | −90.5 | −85.1 | −89.5 | −88.37 |

Ingestible electronics have an expanding role in the valuation of patients. Furthermore, the potential of applying electronics or electrical signals for treatment is being explored, and the potential for long-term monitoring and treatment is being realized through the development of systems with the capacity for safe expanded GI retention. One of the challenges with ingestible systems is the size constraint imposed by ingestion and safe passage through the GI tract. Given these constraints and the limited space available in devices, and furthermore, the potential need for long-term power sources, safe, inexpensive battery alternatives are needed.

The characterization of the ingestible power harvesting device 10 as discussed above is based on an electrochemical cell composed of relatively inexpensive biocompatible materials activated by GI fluid. The ingestible power harvesting device 10 demonstrates energy harvesting from the electrochemical cell for up to 6 days (average power 0.23 μW/mm²). Using this energy, a self-powered device has been developed with the capacity for temperature measurement and wireless transmissions. Furthermore, experiments conducted in the system 98 according to the process 108 demonstrates the capacity of the ingestible power harvesting device 10 for harvesting power from across the GI tract including the stomach, small intestine, and colon. Interestingly, the available power density ranged between a few μW/mm² to a few nW/mm² across the GI tract, with the gastric cavity providing the greatest power density at an average power of 1.14 μW/mm² and an extra gastric power density average noted at 13.2 nW/mm². This observation, specifically the significant difference between gastric and extra gastric density will guide future development of GI resident electronic power harvesting systems according to their targeted anatomic locations. The ingestible power harvesting device 10 could be rapidly implemented for the evaluation of core body temperature and for the evaluation of GI transit time given the different temperatures between the body and the external environment.

Research in ultra-low-power electronics continues to push the boundaries of the average power consumption of devices and already provides a range of options for circuits that could be adapted for GI applications much below 0.23 μW/mm² of power (1 mm² of electrode area), for example, energy harvesters (for sub 10 nW available power), analog-to-digital converters (ADCs), signal acquisition circuits (under 10 nW), far field wireless transmitters (under 1 nW standard power), and mm-scale sensor nodes with sensing and processing (7.7 μW active, 1 nW standby).

Such systems could enable broad applications for extended power harvesting from alternative cells for long-term monitoring of vital signs and other parameters in the GI tract, especially with the introduction of devices that are deployed endoscopically or self-administered and have the capacity to reside in the gastric cavity for a prolonged period of time.

The cathode electrode 12 and the anode electrode 14 of FIG. 1A are created for pure metal foils (Alfa Aesar, 0.25 nm thick) and cut to the specified length and width dimensions to within ±10%. Attachment of the zinc and copper electrodes to wires or to printed circuit boards (PCBs) is performed with standard solder and flux.

All experiments are conducted in accordance with the protocols approved by the Massachusetts Institute of Technology (MIT) Committee of Animal Care. In vivo porcine studies are performed in female Yorkshire pigs weighing approximately 45-50 Kilograms (Kg). Prior to endoscopy or administration of the ingestible power harvesting device 10, the animals are placed on a liquid diet for 48 hours. The animals are fasted overnight immediately prior to the procedure. On the day of the procedure for the endoscopic characterization studies, the animals receive induction of anesthesia with intramuscular injection of Telazol (tiletamine/zolazepam) 5 mg/Kg, xylazine 2 mg/Kg, and atropine (0.04 mg/Kg). The pigs are intubated and maintained on inhaled isoflurane 1-3%. For the deployment of the ingestible power harvesting device 10, the animals are sedated with the intramuscular injections as noted above. The esophagus is intubated and an esophageal overtube placed (US Endoscopy). The ingestible power harvesting device 10 is delivered directly to the gastric cavity or endoscopically placed in the small intestine through the overtube. The ingestible power harvesting device 10 is followed with serial X-rays. A total of 5 stomach-deposited ingestible power harvesting devices are evaluated in 5 separate pig experiments.

A commercial RF transceiver evaluation board (SmartRF TrxEB, Texas Instruments) is used as the ex vivo RF transceiver 104 in the system 98 to receive the plurality of formatted data packets 36 transmitted from the RF transceiver 30 based on 900 MHz frequency-shift keying (FSK). The ex vivo RF transceiver 104 and its respective antenna are mounted above the steel cage area that houses the animals (about 2 meters above the ground). The ex vivo RF transceiver 104 is connected via a universal serial bus (USB) cable to the PC 106 that saves the plurality of formatted data packets 36 for offline processing in MATLAB®.

Those skilled in the art will recognize improvements and modifications to the preferred embodiments of the present disclosure. All such improvements and modifications are considered within the scope of the concepts disclosed herein and the claims that follow.

What is claimed is:

1. An ingestible power harvesting device, comprising:
a cathode electrode and an anode electrode configured to catalyze a power generating reaction to generate a direct current (DC) power between the cathode electrode and the anode electrode in response to being surrounded by an acidic electrolyte; and
an encapsulated electronic device comprising:
power harvesting circuitry coupled to the cathode electrode and the anode electrode, the power harvesting circuitry configured to:
harvest the DC power generated between the cathode electrode and the anode electrode; and
output a DC supply voltage based on the harvested DC power for a prolonged period; and
an electronic circuit powered by the DC supply voltage and configured to support a defined in vivo operation.

2. The ingestible power harvesting device of claim 1, configured to be deployed in a gastrointestinal (GI) tract, wherein the cathode electrode and the anode electrode are configured to catalyze the power generating reaction to generate the DC power in response to being surrounded by gastric acid in the GI tract.

3. The ingestible power harvesting device of claim 1, wherein the electronic circuit further comprises control circuitry powered by the DC supply voltage and configured to control the electronic circuit to carry out the defined in vivo operation.

4. The ingestible power harvesting device of claim 3, wherein the electronic circuit comprises a radio frequency (RF) transceiver configured to transmit information related to the defined in vivo operation via an embedded antenna.

5. The ingestible power harvesting device of claim 4, wherein:
the RF transceiver is further configured to receive commands related to the defined in vivo operation and provide the received commands to the control circuitry; and
the control circuitry is further configured to control the electronic circuit to support the defined in vivo operation based on the received commands.

6. The ingestible power harvesting device of claim 3, wherein:
the encapsulated electronic device is coupled to a drug release system; and
the electronic circuit further comprises a drug release controller configured to control the drug release system to provide controlled in vivo drug release from the drug release system.

7. The ingestible power harvesting device of claim 3, wherein the electronic circuit further comprises a video sensor configured to support in vivo video capture.

8. The ingestible power harvesting device of claim 3, wherein the electronic circuit further comprises a potential-of-hydrogen (pH) sensor configured to support in vivo pH measurement.

9. The ingestible power harvesting device of claim 3, wherein the electronic circuit further comprises a temperature sensor configured to support in vivo temperature measurement.

10. The ingestible power harvesting device of claim 3, wherein the electronic circuit further comprises a pressure sensor configured to support in vivo pressure measurement.

11. The ingestible power harvesting device of claim 3, wherein the electronic circuit further comprises a heartrate sensor configured to support in vivo heartrate measurement.

12. The ingestible power harvesting device of claim 3, wherein the electronic circuit further comprises a respiration sensor configured to support in vivo respiration measurement.

13. The ingestible power harvesting device of claim 1, wherein:
the cathode electrode is a copper metal electrode; and
the anode electrode is a zinc electrode.

14. The ingestible power harvesting device of claim 1, wherein the encapsulated electronic device is encapsulated by silicone.

15. The ingestible power harvesting device of claim 1, wherein the power harvesting circuitry is further configured to output the DC supply voltage to the electronic circuit periodically.

16. The ingestible power harvesting device of claim 15, wherein the encapsulated electronic device further comprises:
a capacitor coupled to the power harvesting circuitry; and
a metal-oxide semiconductor field-effect transistor (MOSFET) switch disposed between the capacitor and the electronic circuit;
wherein the MOSFET switch is configured to:
couple the power harvesting circuitry to the electronic circuit in response to the DC supply voltage being higher than or equal to a threshold voltage of the MOSFET switch; and
decouple the power harvesting circuitry from the electronic circuit in response to the DC supply voltage being lower than the threshold voltage of the MOSFET switch.

17. A method for evaluating average power harvested by an ingestible power harvesting device, comprising:
deploying an ingestible power harvesting device in a porcine gastrointestinal (GI) tract, wherein the ingestible power harvesting device comprises:
a cathode electrode and an anode electrode configured to catalyze a power generating reaction to generate a direct current (DC) power between the cathode electrode and the anode electrode in response to being surrounded by an acidic electrolyte; and
an encapsulated electronic device comprising:
power harvesting circuitry coupled to the cathode electrode and the anode electrode, the power harvesting circuitry configured to harvest the DC power and output a DC supply voltage based on the harvested DC power; and
a radio frequency (RF) transceiver;
transmitting a plurality of formatted data packets from the RF transceiver within a predetermined duration;
receiving the plurality of formatted data packets at an ex vivo RF transceiver located within an RF coverage range of the RF transceiver; and
determining an average DC power harvested by the power harvesting circuitry in the predetermined duration based on a count of formatted data packets received at the ex vivo RF transceiver and power consumption associated with transmitting each of the plurality of formatted data packets.

18. The method of claim 17, further comprising:
embedding a packet counter in each of the plurality of formatted data packets transmitted from the RF transceiver; and
determining the count of the formatted data packets received at the ex vivo RF transceiver based on a maximum packet counter value conveyed in the plurality of formatted data packets received by the ex vivo RF transceiver.

19. The method of claim 17, further comprising determining the power consumption associated with transmitting each of the plurality of formatted data packets by measuring power consumed for transmitting an experimental data packet having an identical packet length as a formatted data packet in a laboratory experiment.

20. The method of claim 17, further comprising:
receiving commands related to a defined in vivo operation from the ex vivo RF transceiver; and
controlling the encapsulated electronic device based on the received commands.
